(19) United States
(12) Patent Application Publication
(10) Pub. No.: US 2018/0161194 A1
(43) Pub. Date: Jun. 14, 2018

(54) JAW FIXATION BELT

(71) Applicant: IKK CO., LTD., Kahoku-shi, Ishikawa (JP)

(72) Inventors: Setsuo TAKIZAWA, Nomi-shi, Ishikawa (JP); Kazuhide ITO, Kahoku-shi, Ishikawa (JP)

(21) Appl. No.: 15/578,040

(22) PCT Filed: Sep. 29, 2016
(86) PCT No.: PCT/JP2016/078769

§ 371 Date: Nov. 29, 2017

(30) Foreign Application Priority Data
Nov. 18, 2015 (JP) 2015-225260
Apr. 7, 2016 (JP) 2016-077000

(51) Int. Cl. A61F 5/058; A61F 5/56; A61B 90/14; A61F 5/24; A63B 71/10

(52) U.S. Cl. CPC A61F 5/05891; A61F 5/56; A63B 71/10; A61F 5/24; A61B 90/14

(57) ABSTRACT

A jaw fixation belt is gas permeable and comfortable to wear even in summer, can be securely fixed to the head even if the head or body moves when the wearer rolls over while asleep, for example, and is stretchable and properly fitted to the face. A jaw fixation belt according to the present invention, which is worn extending from the top of the head to the jaw, is provided with a first jaw fastening belt that fastens the jaw from the front side of the chin; and a second jaw fastening belt that fastens the jaw from the rear side of the chin, wherein a chin fixing portion in which one edge of the center of the first jaw fastening belt and the other edge of the center of the second jaw fastening belt are joined to each other is provided.

### TECHNICAL FIELD

The present invention relates to a jaw fixation belt for medical purposes used to prevent snoring or sleep apnea syndrome resulting from breathing with opened mouth during sleep, or used for a gastroscopy examination by an endoscope or endoscopic surgery of stomach or esophagus over a long time, and is capable of enhancing the muscle around jaw by fixing the jaw to obtain a small face effect.

### BACKGROUND ART

Conventionally, a jaw fixation belt that is used by hanging between the jaw and the top of the head has been used in various fields to prevent snoring, sleep apnea syndrome, and the like, and as a medical belt for fixing the jaw used for stomach camera inspection by endoscope or an endoscopic surgery of stomach or esophagus over a long time, and even as a supporter used during sports or martial arts, or a belt for cosmetic purposes to obtain a small face effect.

However, conventional jaw fixation belts (FIG. 12) readily get stuffy due to bad air permeability when being worn over a long time, and can be displaced and detached by the motion of the face, such as rolling over while sleeping because of inferior in their stretch. Furthermore, by the problem of durability of the jaw fixation belt itself, there is a problem in that the jaw fixation belt is disadvantageously elongated during being used to become unusable.

Patent Literature 1 relates to a jaw band to prevent snoring during sleep, and describes that it is formed of an annular band to be hung between the jaw and the top of the head, and made of a knit, and a portion to be overlaid on the jaw and a portion to be overlaid on the top of the head have a wide range of about palm size (paragraph 0024 therein), and the portion to be overlaid on the jaw is made of a knit formed of assembly of reticulation (knitted fabric), so that the portion overlaid on the jaw is expanded and contracted along the shape of the jaw to be fitted well to the jaw, making it difficult to be detached during sleep (paragraphs 0016, 0018 therein).

Patent Literature 2 relates to a head fitting tool for preventing snoring or apnea, and describes that the head fitting tool includes: a bar like elastic body vertically disposed at the back of the head of the user with a predetermined length; a holding sheet to which the bar like elastic body is attached; a band like body attached to surround a circumference of the holding sheet; a pair towing bodies attached at respective positions of the band like body on both sides of the facial surface with a predetermined gap, and a stretchable jaw holding member attached to be stretched between the pair of towing bodies (paragraph 0019 therein), and the holding sheet is formed of a net like sheet, so that the tool has air permeability as well as stiffness (size stability) to stick fast to the head of user to prevent uncomfortable feeling due to sweating or the like during sleep (paragraph 0024 therein).

Furthermore, Patent Literature 3 relates to a snoring suppression tool that suppresses snoring by wearing the tool during sleep, and the tool is comprised of a lower jaw holding band part and an upper jaw holding band part, and the lower jaw holding band part is formed in an annular shape, and is worn by hanging between the top of the head and the lower jaw (paragraph 0015 therein). It describes that forming the lower jaw holding band part and the upper jaw holding band part by a material formed of, for example, polyester, polyurethane, or the like having a soft texture feeling that is mild to a person as well as elasticity results in a texture feeling having fit feel, and does not cause discomfort even when a person wears the snoring suppression tool of the invention over a long time during sleep (paragraph 0010 therein).

### CITATION LIST

### Patent Literature

PTL 1: JP-A-2001-299791
PTL 2: JP-A-2013-233188
PTL 3: Japanese Utility Model No. 3191180

### SUMMARY OF INVENTION

### Technical Problem

However, in order to prevent a jaw fixation band from being displaced to the head in response to the motion of the face such as rolling over while sleeping, it is effective to fix the chin to be sandwiched from the front side and back side of the lower jaw, whereas the structure of the jaw band, the whole of which is formed of a knit, disclosed in Patent Literature 1 fails to sufficiently sandwich the chin, and elasticity of the jaw band are large, so that there is a merit in that the portion of the face or the head to be in contact with the jaw band does not feel pain even when the jaw band is worn over a long time, but conversely the effect of fixing the chin is reduced to cause displacement or detachment. Furthermore, although the snoring suppression tool disclosed in Patent Literature 3 can fix the jaw by the lower jaw holding band part and the upper jaw holding band part and suppress getting stuffy due to sweating because of its mesh structure, the whole of which is formed of the same material similar to Patent Literature 1, so that the snoring suppression tool causes pain at a portion of the face or the head to be in contact therewith when being firmly tightened by a hook-and-loop fastener to sufficiently fix the tool to the head, whereas the tool is readily detached from the head when being loosely tightened, conversely.

Furthermore, in the head fitting tool disclosed in Patent Literature 2, although the lower jaw can be fixed by fixing the jaw holding member to the pair of towing bodies to have an appropriate predetermined length, the structure becomes complex and its manufacturing cost increases because of using a number of members.

Therefore, the object of the invention is to provide a jaw fixation belt that has air permeability so that it can be comfortably worn even in summer, that is firmly fixed to the head even when the motion of the head or the body such as rolling over while sleeping occurs, and that stretches well so that it can be appropriately worn on the face.

Furthermore, the object of the invention is to provide a highly durable jaw fixation belt that has a simple structure, that is easy in manufacturing, and that is long lasting even when used many times.

### Solution to Problem

In the invention, a jaw fixation belt worn by hanging it between the top of the head and the jaw includes a first jaw tightening belt for tightening a jaw from a front side of a chin, and a second jaw tightening belt for tightening the jaw from a back side of the chin, and has a chin fixation portion in which one edge of a central part of the first jaw tightening belt and another edge of a central part of the second jaw tightening belt are joined.

The Jaw fixtures of Patent Literature 1, Patent Literature 2, and Patent Literature 3, which are conventional techniques, cause pain at a portion of the face or the head to be in contact therewith when the jaw fixtures are firmly tightened to sufficiently fix to the head, and causes pain at the chin or the like because force is concentrated at one portion such as the chin in the portion of the jaw where the tools are fixed.

According to the invention, when the first jaw tightening belt is applied to the front side of the chin, the second jaw tightening belt is applied to the back side of the chin, and the first jaw tightening belt and the second jaw tightening belt are pulled upward, since the one edge of the central part of the first jaw tightening belt and the other edge of the central part of the second jaw tightening belt are joined, force works in directions to bring another edge of the first jaw tightening belt and one edge of the second jaw tightening belt close, and the whole structure firmly sandwiches the chin, resulting in comfortable and firm fixation without feeling pain at the jaw.

Furthermore, in the jaw fixation belt of the invention, the first jaw tightening belt and the second jaw tightening belt are formed of mesh fabric that is an elastic narrow-width knit created by a covering yarn whose core is a rubber-based or polyurethane elastic yarn.

According to the invention, the first jaw tightening belt and the second jaw tightening belt are formed of mesh fabric that is an elastic narrow-width knit created by a covering yarn whose core is rubber-based or polyurethane elastic yarn, it is thus possible to provide a jaw fixation belt that does not elongates or cuts even when used many times, that can be firmly fixed to the head without being displaced or detached even when the motion of the head or the body such as rolling over occurs, and that can be comfortably worn by the user over a long time. Furthermore, it becomes possible to provide a jaw fixation belt having high durability by exercising an ingenuity that makes difficult to occur fraying at the edges by using a narrow-width knit knitted in narrow manner for the jaw fixation belt makes it.

Furthermore, according to the invention, the jaw fixation belt is mainly formed of the first jaw tightening belt and the second jaw tightening belt. Therefore, its structure is simple and it is easy in manufacturing.

The jaw fixation belt of the invention further includes face-end mounting units to be fitted to both ends of a face formed of the first jaw tightening belt and/or the second jaw tightening belt.

When the whole jaw fixation belt is formed in the same stretch rate, it is impossible to adjust stretch rate depending on the wearing portion. Therefore, the problem occurs in which when the fixing force by the jaw fixation belt is strong a fixing force to be applied on the portion other than the intended fixation place (jaw) becomes too strong, which makes the wearer feel uncomfortable, and conversely, when the jaw fixation belt is loosely tightened it readily comes off from the head.

Thus, according to the invention, a stretch force in the face-end mounting units to be worn at both ends of the face is changed/adjusted with respect to the places other than that in manufacturing stage by reduplicating the first jaw tightening belt and the second jaw tightening belt, or by using one of the first jaw tightening belt and the second jaw tightening belt as the face-end mounting units, in order to provide a jaw fixation belt that can be comfortably worn without causing the wearer to feel pain at both sides of the face.

In the jaw fixation belt of the invention, a first cloth piece is provided at the one edge of the central part of the first jaw tightening belt, a second cloth piece is provided at the other edge of the central part of the second jaw tightening belt, and the chin fixation portion is formed by joining the first cloth piece and the second cloth piece.

Furthermore, in the jaw fixation belt of the invention, the first cloth piece and the second cloth piece are formed of a material having a stretch rate smaller than that of the first jaw tightening belt and the second jaw tightening belt.

Herein, “stretch rate” denotes stretch rate during constant load when load is 0.5 kg described in JIS L1096. Herein, the stretch rate when no load is applied shall be 100%, and the stretch rate can be expressed by the following Expression 1.

S=100×B/A Expression 1

Herein, S is stretch rate [%], B is the length of cloth when load is 0.5 kg, and A represents the length of cloth when no load is applied.

In order to prevent a jaw fixation belt from being displaced to the head in response to the motion of the face such as rolling over, it is effective to fix the chin to be sandwiched from the front side and back side of the lower jaw, whereas when a material having high stretchability is used as the jaw fixation belt, there is a merit in that a portion of the face or the head to be in contact with the jaw fixation belt does not feel pain even when the jaw fixation belt is worn over a long time, but conversely the effect of fixing the chin is reduced, resulting in displacement or detachment.

According to the invention, a jaw fixation belt that can be comfortably worn by the user over a long time is provided by forming with mesh fabric that is an elastic narrow-width knit in its entirety, and the chin fixation portion to be worn on the chin that is an intended fixation place is made not to expand and contract more than other places by using a first cloth piece and a second cloth piece that are a material having a stretch rate smaller than that of the first jaw tightening belt and the second jaw tightening belt to, resulting in firm fixation of the chin. By changing the stretch rate of the chin fixation portion to be worn on the chin that is intended fixation place, it becomes possible to provide a jaw fixation belt that makes the user comfortable and capable of being firmly fixed to the chin.

In the jaw fixation belt of the invention, a third cloth piece is provided at another edge of the center of the first jaw tightening belt, and a fourth cloth piece is provided at one edge of the central part of the second jaw tightening belt.

Furthermore, in the jaw fixation belt of the invention, the third cloth piece and the fourth cloth piece are formed of a material having a stretch rate smaller than that of the first jaw tightening belt and the second jaw tightening belt.

According to the invention, the stretch rate of the third cloth piece provided at the other edge of the central part of the first jaw tightening belt and the fourth cloth piece provided at the one edge of the central part of the second jaw tightening belt is smaller than that of the first jaw tightening belt and the second jaw tightening belt, so that the chin fixation portion is prevented from being expanded and contracted by the first cloth piece, the second cloth piece, the third cloth piece, and the fourth cloth piece and more firmly fixes the chin, as well as the face-end mounting units expand and contract and is fixed to the head without being displaced and detached even when the head or the body moves.

In the jaw fixation belt of the invention, the first jaw tightening belt and the second jaw tightening belt consist of a band-like member comprised of mesh fabric in which a large number of holes are formed, and the mesh fabric has a stretch rate of 120 to 300%.

According to the invention, since the jaw fixation belt consists of a band-like member comprised of mesh fabric in which a large number of holes are formed, air permeability is good and use feeling is excellent, and moreover, since its stretch rate is made to be 120 to 300%, its elasticity becomes good, the belt is tightly fixed to the head without being displaced and detached even when the head or the body moves.

The jaw fixation belt of the invention is further provided with a pair of displacement prevention belts attached at a place where the central part of the first jaw tightening belt and the central part of the jaw fixation belt are joined.

Furthermore, the jaw fixation belt of the invention is further provided with a pair of coupling belts provided between the first jaw tightening belt and the displacement prevention belt and between the second jaw tightening belt and the displacement prevention belts, respectively.

According to the invention, the jaw fixation belt is fixed to the back of head by providing the pair of displacement prevention belts and the pair of coupling belts in addition to the jaw fixation belt, increasing fixing force to the head.

Specifically, since the displacement prevention belts are attached at the places where the central part of the first jaw tightening belt and the central part of the second jaw tightening belt are joined, when being pulled in the back direction of the wearer, the whole first jaw tightening belt of the chin fixation portion is firmly pressed against the front side of the chin, achieving a remarkable function effect that the chin fixation is fixed to the chin more strongly. The function effect is generated by the fact that the displacement prevention belts are attached at the places where the central part of the first jaw tightening belt and the central part of the second jaw tightening belt are joined.

In the jaw fixation belt of the invention, the jaw fixation belt is provided with a hole for attaching a mask used for treatment of sleep apnea syndrome.

According to the invention, the jaw fixation belt includes a pair of holes for inserting therein a belt of a fixture for wearing a respirator in respective the pair of face-end mounting units provided in the jaw fixation belt. Therefore, it possible to insert the belt into the holes to comfortably use the jaw fixation belt in conjunction with a mask used for treatment of sleep apnea syndrome.

The jaw fixation belt of the invention can be used in various fields as a belt for medical purpose for preventing snoring or preventing sleep apnea syndrome or the like, as a supporter used during sports or martial arts, or as a belt for cosmetic purposes to obtain a small face effect.

### Advantageous Effects of Invention

According to the invention, when the first jaw tightening belt is applied to the front side of the chin, the second jaw tightening belt is applied to the back side of the chin, and the first jaw tightening belt and the second jaw tightening belt are pulled upward, since the one edge of the central part of the first jaw tightening belt and the other edge of the central part of the second jaw tightening belt are joined, force works in directions to bring another edge of the first jaw tightening belt and one edge of the second jaw tightening belt close, and the whole structure firmly sandwiches the chin, resulting in comfortable and firm fixation without feeling pain at the jaw.

Also, according to the invention, the first jaw tightening belt and the second jaw tightening belt are formed of mesh fabric that is an elastic narrow-width knit created by a covering yarn whose core is rubber-based or polyurethane elastic yarn, it is thus possible to provide a jaw fixation belt that does not elongates or cuts even when used many times, that can be firmly fixed to the head without being displaced or detached even when the motion of the head or the body such as rolling over occurs, and that can be comfortably worn by the user over a long time. Furthermore, it becomes possible to provide a jaw fixation belt having high durability by exercising an ingenuity that makes difficult to occur fraying at the edges by using a narrow-width knit knitted in narrow manner for the jaw fixation belt makes it.

Furthermore, according to the invention, the jaw fixation belt is mainly formed of the first jaw tightening belt and the second jaw tightening belt. Therefore, its structure is simple and it is easy in manufacturing.

In addition, according to the invention, the first, second, third and fourth cloth pieces provided at the edges of the central parts of the first and second jaw tightening belts are smaller than that of the first and second jaw tightening belts, so that the chin fixation portion is less expanded and contracted than the face-end mounting units by effect of the first, the second, the third and fourth cloth pieces and more firmly fixes the chin, as well as the face-end mounting units expand and contract and is fixed to the head without being displaced and detached even when the head or the body moves.

Finally, according to the invention, since the jaw fixation belt consists of a band-like member comprised of mesh fabric in which a large number of holes are formed, air permeability is good and use feeling is excellent, and moreover, since its stretch rate is made to be 120 to 300%, its elasticity becomes good, the belt is tightly fixed to the head without being displaced and detached even when the head or the body moves.

### BRIEF DESCRIPTION OF DRAWINGS

FIG. 1 is a perspective view illustrating a jaw fixation belt 10 of a first embodiment to which the invention is applied.

FIG. 2 is a schematic view of a state where the jaw fixation belt 10 of the above embodiment is spread.

FIG. 3 is a B-B cross-sectional view of the jaw fixation belt 10 of the above embodiment.

FIG. 4 is an enlarged outside view of a central part of the jaw fixation belt 10 of the above embodiment.

FIG. 5 is a C-C cross-sectional view of the jaw fixation belt 10 of the above embodiment.

FIG. 6 is an E-E cross-sectional view of the jaw fixation belt 10 of the above embodiment.

FIG. 7 is an enlarged inside view of the central part of the jaw fixation belt 10 of the above embodiment.

FIG. 8 is a perspective view of a state where the jaw fixation belt 10 of the above embodiment is worn.

FIG. 9 is a front view of a state where the jaw fixation belt 10 of the above embodiment is worn.

FIG. 10 is an enlarged view in which first and second jaw tightening belts used in the jaw fixation belt 10 of the above embodiment are enlarged.

FIG. 11 is a C-C cross-sectional view when the jaw fixation belt 10 of the above embodiment is worn.

FIG. 12 is a photograph showing conventional jaw fixation belts 200, 300, 400.

FIG. 13 is a schematic view of a state where a jaw fixation belt 20 of a second embodiment to which the invention is applied is spread.

FIG. 14 is a perspective view of a state where the jaw fixation belt 20 of the above embodiment is worn.

FIG. 15 is a perspective view illustrating a jaw fixation belt 30 of a third embodiment to which the invention is applied.

FIG. 16 is a schematic view of a state where the jaw fixation belt 30 of the above embodiment is spread.

FIG. 17 is a perspective view illustrating another example 40 of the jaw fixation belt of the first embodiment to which the invention is applied.

FIG. 18 is a development schematic view of the other example 40 of the jaw fixation belt of the above embodiment.

### DESCRIPTION OF EMBODIMENTS

Embodiments of the invention will be described in detail using the drawings

### First Embodiment of the Invention

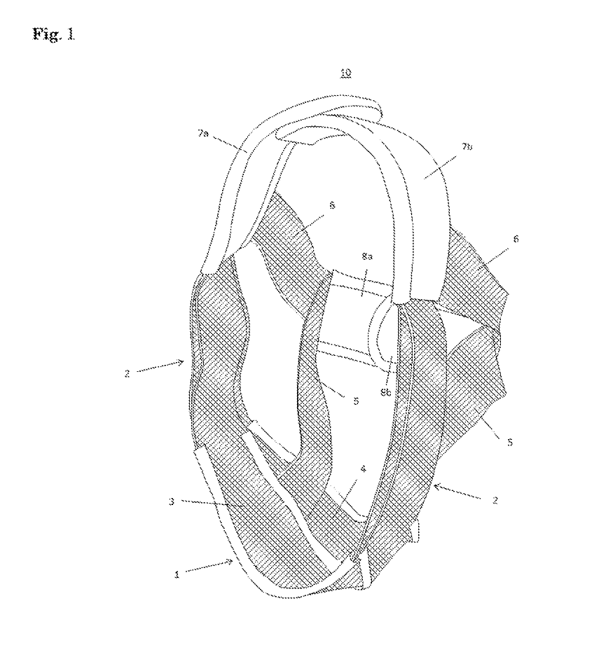

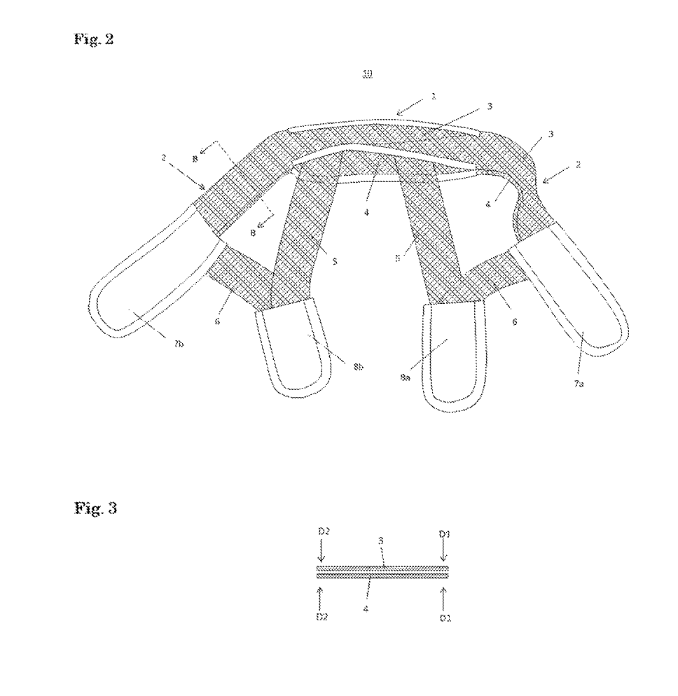

FIG. 1 is a perspective view illustrating a jaw fixation belt 10 of a first embodiment to which the invention is applied, and FIG. 2 is a schematic view of a state where the jaw fixation belt 10 of the above embodiment is spread. FIG. 3 is a B-B cross-sectional view of the jaw fixation belt 10 of the above embodiment.

The jaw fixation belt 10 of the embodiment consists of a pair of face-end mounting units 2, 2, a chin fixation portion 1, detachable members 7a, 7b provided at respective ends of the face-end mounting units 2, 2, a pair of displacement prevention belts 5, 5, displacement prevention belt detachable members 8a, 8b provided at respective ends of the displacement prevention belts 5, 5, and a pair of coupling belts 6, 6.

The pair of face-end mounting units 2, 2, and the chin fixation portion 1 are formed by joining ends of a first jaw tightening belt 3 and ends of a second jaw tightening belt 4 by thermo-compression bonding, sewing, bonding, etc.

The pair of face-end mounting units 2, 2 are formed by utilizing ends of the first jaw tightening belt 3 and the second jaw tightening belt 4, and the chin fixation portion 1 is formed by utilizing central parts of the first jaw tightening belt 3 and the second jaw tightening belt 4. Therefore, joining places of the pair of face-end mounting units 2, 2 and the chin fixation portion 1 are consecutively formed. In this manner, the jaw fixation belt 10 of the embodiment is mainly formed of the first jaw tightening belt and the second jaw tightening belt, therefore, its structure is simple and it is easy in manufacturing.

The pair of surface-end mounting units 2, 2 are sites to be in contact with both ends of the face, and are doubly formed by joining the first jaw tightening belt 3 and the second jaw tightening belt 4 in such a manner that edges D1 and edges D2 are overlapped each other such that each pair of their ends are not displaced (FIG. 3). Furthermore, the detachable members 7a, 7b are attached to respective one ends of the first jaw tightening belt 3 and the second jaw tightening belt 4 (one ends of the face-end mounting units 2, 2). Furthermore, the other ends of the face-end mounting units 2, 2 are integrally formed with respective ends of the chin fixation portion 1.

The joining places of the first jaw tightening belt 3 and the second jaw tightening belt 4 may be one edges D1 and the other edges D2 of the first jaw tightening belt 3 and the second jaw tightening belt 4 (FIG. 3). Alternatively, the first jaw tightening belt 3 and the second jaw tightening belt 4 may be joined at the joining places of the pair of face-end mounting units 2, 2 and the respective detachable members 7a, 7b instead of the both edges.

The detachable members 7a, 7b are members for detachably wearing the jaw fixation belt 10 on the head, and are provided at respective ends of the face-end mounting units 2, 2. When the detachable member 7a, 7b are overlapped and adhered, the first jaw tightening belt 3 and the second jaw tightening belt 4 form an annular shape to be fixed to the head, and when the detachable members 7a, 7b are detached, the first jaw tightening belt 3 and the second jaw tightening belt 4 form an elongated shape, making it possible to be detached from the head. As the detachable members 7a, 7b, for example, a hook-and-loop fastener, a buckle, a hook, and the like in which a male member 7a and a female member 7b are detachable at their facing surfaces are appropriately selectable as long as the circumference length of the jaw fixation belt 10 when it is formed in an annular shape is adjustable.

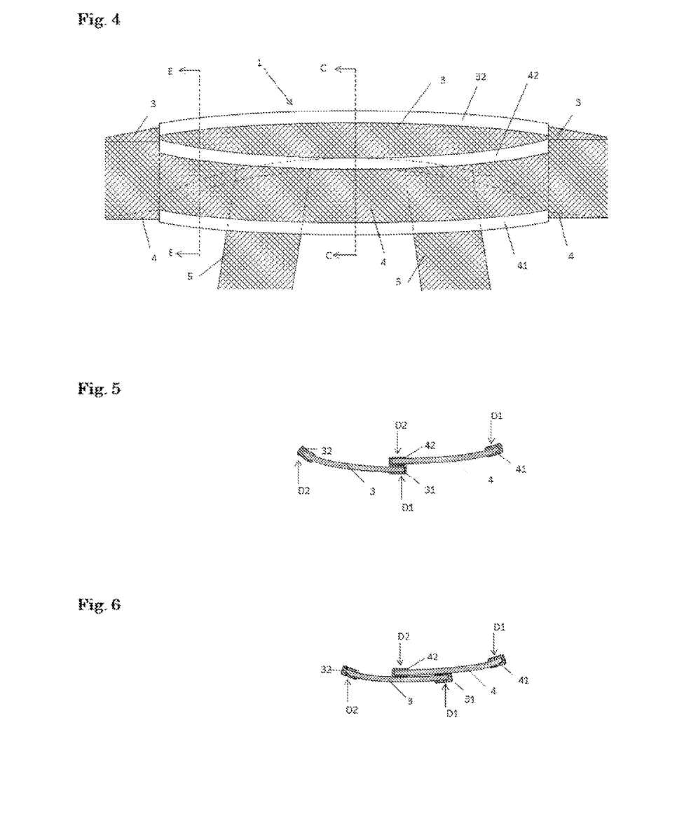

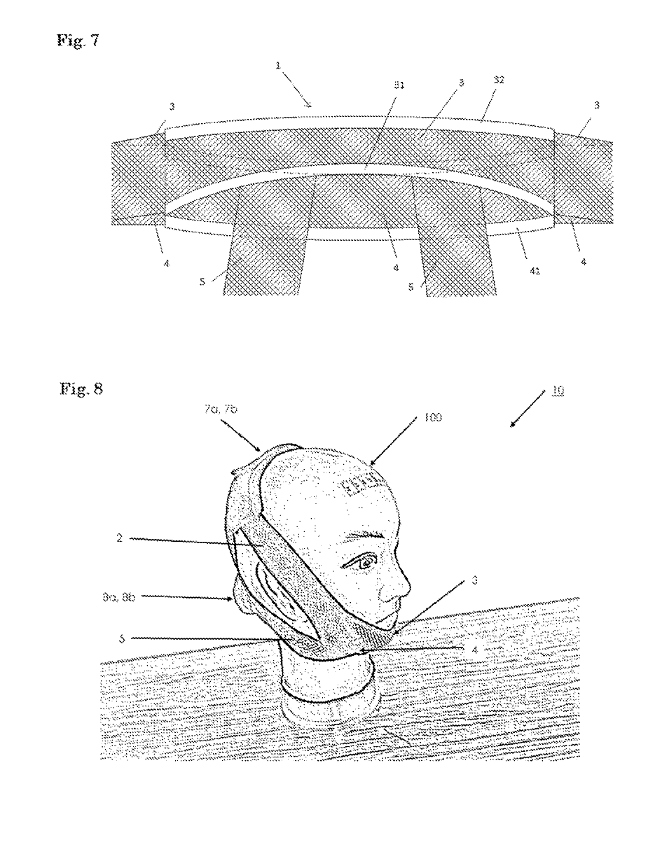

FIG. 4 is an enlarged outside view of a central part of the jaw fixation belt 10 of the above embodiment, and FIG. 5 is a C-C cross-sectional view of the jaw fixation belt 10 of the above embodiment. FIG. 6 is an E-E cross-sectional view of the jaw fixation belt 10 of the above embodiment, and FIG. 7 is an enlarge inside view of the central part of the jaw fixation belt 10 of the above embodiment.

The chin fixation portion 1 is a portion for sandwiching the chin for fixation, and in the center C-C of the chin fixation portion 1 (FIG. 4), one edge D1 of the central part of the first jaw tightening belt 3 and the other edge D2 of the central part of the second jaw tightening belt 4 are stacked and joined, and the first jaw tightening belt 3 and the second jaw tightening belt 4 are joined such that their stacking area is gradually increased from the center toward the endmost portions of the chin fixation portion 1 (FIG. 4, FIG. 6). At the endmost portions of the chin fixation portions 1, the first jaw tightening belt 3 and the second jaw tightening belt 4 are joined in such a manner that edges D1 and edges D2 are overlapped with each other so as not to be substantially displaced. Joining the first jaw tightening belt 3 and the second jaw tightening belt 4 in this manner makes the chin fixation portion 1 have a three-dimensional shape to confine.

At both edges D1, D2 of the central part of the first jaw tightening belt 3, a first cloth piece 31 and a third cloth piece 32 are respectively provided by being folded in two, made to respectively sandwich both edges D1, D2 from their outer sides, and joined by thermo-compression bonding, sewing, bonding, etc., and also at both edges D1, D2 of the central part of the second jaw tightening belt, in the same manner, a fourth cloth piece 41 and a second cloth piece 42 are respectively provided by being folded in two, made to respectively sandwich both edges D1, D2 from their outer sides, and joined by thermo-compression bonding, sewing, bonding, etc.

The first cloth piece 31, the third cloth piece 32, the fourth cloth piece 41, the second cloth piece 42 are provided to prevent the chin fixation portion 1 from being too elongated to appropriately keep the shape of the chin fixation portion 1, formed by making a yarn made of polyester, nylon, or the like have a band shape with a loom, a braiding machine, or the like, and formed of a material having a stretch rate smaller than those of the first jaw tightening belt 3 and the second jaw tightening belt 4.

The first cloth piece 31, the third cloth piece 32, the fourth cloth piece 41, the second cloth piece 42 may not be folded in two and made to sandwich both edges D1, D2 from their outer sides, and at both ends D1, D2 of the first and second tightening belts 3, 4, cloth pieces may be respectively provided on upper and lower sides in the paper (FIG. 5, FIG. 6) and made to join to the both ends D1, D2, or a cloth piece may be provided on only one of upper and lower sides in the paper (FIG. 5, FIG. 6) and made to join to the both ends D1, D2.

Only the first cloth piece 31 and the second cloth piece 42 may be provided on both edges D1, D2 of the central parts of the first and second tightening belts 3, 4, or only the third cloth piece 32 and the fourth cloth piece 41 may be provided. By providing the first cloth piece 31 the third cloth piece 32, the fourth cloth piece 41, and the second cloth piece 42 makes it possible to prevent the chin fixation portion 1 from being too elongated, it becomes possible to adequately keep the shape of the chin fixation portion 1 to firmly fix the chin.

The pair of displacement prevention belts 5, 5 is for appropriately fixing the chin fixation belt 10 at the back of the head, and one ends of the pair of displacement prevention belts 5, 5 are attached with a space therebetween to the place where the central part of the first jaw tightening belt and the central part of the second jaw tightening belt are joined (FIG. 4, FIG. 7), and displacement prevention belt detachable members 8a, 8b are attached to respective the other ends thereof.

The displacement prevention belt detachable members 8a, 8b are members for detachably wearing the pair of displacement prevention belts 5, 5 on the back of the head. When the displacement prevention belt detachable members 8a, 8b are overlappingly adhered with each other the pair of displacement prevention belts 5, 5 are fixed to the back of the head, and when the displacement prevention belt detachable members 8a, 8b are detached the pair of displacement prevention belts 5, 5 can be detached from the back of the head. As the displacement prevention belt detachable members 8a, 8b, for example, a hook-and-loop fastener, a buckle, a hook, and the like in which the male member 8a and the female member 8b are detachable at their facing surfaces are appropriately selectable.

The pair of the coupling belts 6, 6 is for coupling the pair of face-end mounting units 2, 2 and the pair of displacement prevention belts 5, 5 to prevent the pair of face-end mounting units 2, 2 and the pair of displacement prevention belts 5, 5 from being displaced on the head when the jaw fixation belt 10 is worn on the head. One ends of the pair of coupling belt 6, 6 are attached to respective the joining places of the pair of displacement prevention belts 5, 5 and the displacement prevention belt detachable members 8a, 8b and the other ends thereof are attached to respective the detachable members 7a, 7b or respective ends of the pair of face-end mounting units 2, 2 (FIG. 2).

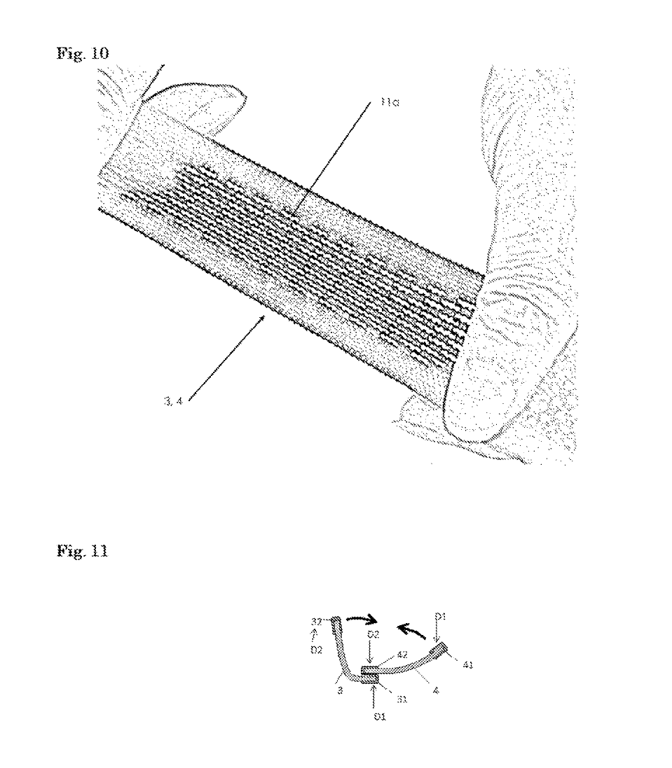

FIG. 10 is an enlarged view enlarging the first and second jaw tightening belts used in the jaw fixation belt 10 of the above embodiment.

The first jaw tightening belt 3 and the second jaw tightening belt 4 have a long band shape, and formed to be the same size with the same material. Furthermore, the first jaw tightening belt 3 and the second jaw tightening belt 4 are formed of mesh fabric in which a large number of holes 11a are formed, and their stretch rates are increased by the large number of holes 11a.

The first jaw tightening belt 3 and the second jaw tightening belt 4 are formed of mesh fabric that is an elastic narrow-width knit created by a covering yarn whose core is a rubber-based or polyurethane elastic yarn. The covering yarn denotes a yarn having a rubber-based or polyurethane elastic yarn as its core and protecting the yarn by winding therearound a yarn made of polyester or nylon, and the covering yarn is woven into a mesh shape in which the large number of holes 11a are formed by a braiding machine to form mesh fabric.

The covering yarn is superior in elasticity because a rubber based or polyurethane elastic yarn having a high stretch rate is used in its core, and has a high durability because a yarn made of polyester or the like protects the rubber based or polyurethane elastic yarn from above. Likewise, mesh fabric formed of the covering yarn also becomes a cloth superior in elasticity and durability.

Preferably, the stretch rate of the mesh fabric formed of the covering yarn is 120% to 300%.

Furthermore, the elastic narrow-width knit is a knit created by knitting a material having high elasticity to have a required narrow-width in a manufacturing stage, and has an effect that its ends are less likely to be frayed as compared with the case where a wide width knitting is cut to be used.

Although the shape of the large number of holes 11a is a diamond shape in the embodiment, various shapes are selectable such as a rectangular shape, an elliptical shape, a star shape, a square shape, a triangular shape, a polygonal shape, and a round shape, and the large number of holes 11a are provided at intervals in the longitudinal direction.

The displacement prevention belts 5, 5 and the pair of coupling belts 6, 6 are also formed of the material similar to that of the first and second jaw tightening belts 3, 4.

In this manner, since a cloth having elasticity in which the large number of holes 11a are provided, the jaw fixation belt 10 has air permeability, can be comfortably worn even in summer, and firmly fixed to the head even when the motion of the head or the body such as rolling over while sleeping occurs. Furthermore, by providing the large number of holes 11a at intervals in the longitudinal direction, the stretch rate in the longitudinal direction becomes larger, allowing the jaw fixation belt 10 to expand and contract in greater way to adequately fit to the head. Furthermore, since the jaw fixation belt 10 is formed of the covering yarn whose core is a rubber-based or polyurethane elastic yarn, the jaw fixation belt 10 does not elongates or cuts even when the jaw fixation belt 10 is used many times, it is thus possible to provide a jaw fixation belt having a high durability.

Next, a wearing method of the jaw fixation belt of the embodiment will be described.

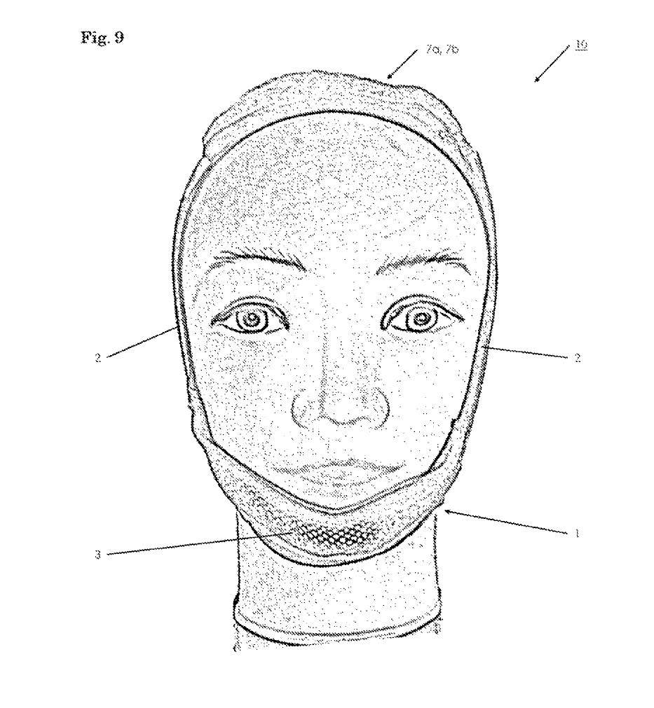

FIG. 8 is a perspective view of a state where the jaw fixation belt 10 of the above embodiment is worn, and FIG. 9 is a front view of a state where the jaw fixation belt 10 of the above embodiment is worn. FIG. 11 is a C-C cross-sectional view when the jaw fixation belt 10 of the above embodiment is worn.

(Step 1)

First, the jaw fixation belt 10 is spread in front of the face of a wearer 100, and the chin fixation portion 1 is lightly applied to the jaw of the wearer 100. Herein, the chin fixation portion 1 is a three-dimensional shape and is pressed such that a front side of the chin is made to contact with the first jaw tightening belt 3 of the chin fixation portion 1, and a back side of the chin is made to contact with the second jaw tightening belt 4 of the chin fixation portion 1.

(Step 2)

Next, the face-end mounting units 2, 2 are made to contact with respective both ends of the face, and the detachable members 7a, 7b are adhered at the top of the head of the wearer 100. Herein, when the detachable members 7a, 7b are adhered while pulling the face-end mounting units 2, 2 upward, the both ends of the first jaw tightening belt 3 and the second jaw tightening belt 4 of the chin fixation portion 1 are pulled upward, so that force works in directions to bring the edges that are not joined of the first jaw tightening belt 3 and the second jaw tightening belt 4 (the other edge D2 of the first jaw tightening belt 3 and the one end D1 of the second jaw tightening belt 4) close (FIG. 11), making it possible to firmly sandwich and sufficiently fix the chin. Furthermore, when the first cloth piece 31, the third cloth piece 32, the fourth cloth piece 41, and the second cloth piece 42 are provided on the edges of the chin fixation portion 1, it is possible that the chin fixation portion 1 does not elongate too much and adequately keeps the shape of the chin fixation portion 1, the chin is thus firmly fixed, and the face-end mounting units 2, 2 can expand and contract to be adequately fixed to the both ends of the face.

(Step 3)

Then, the displacement prevention belts 5, 5 are made to contact with back sides of the ears of the wearer 100, and the displacement prevention belt detachable members 8a, 8b are adhered at the back of the head.

(Step 4)

Then, the adhesion positions of the detachable members 7a, 7b and the displacement prevention belt detachable members 8a, 8b, and the positions of the coupling belts 6, 6 are appropriately adjusted to adjust the fixation conditions to the head of the wearer 100.

In this manner, by using the jaw fixation belt of the invention, it is possible that the chin fixation portion 1 does not elongate too much and appropriately keeps the shape of the chin fixation portion 1 to firmly fix the chin. Furthermore, since the face-end mounting units 2, 2 expand and contract well, the jaw fixation belt is firmly fixed to the head without being displaced or detached even when the motion of the head or the body such as rolling over while sleeping occurs, making it possible to keep a mouth closed state over a long time.

### Another Example of First Embodiment of the Invention

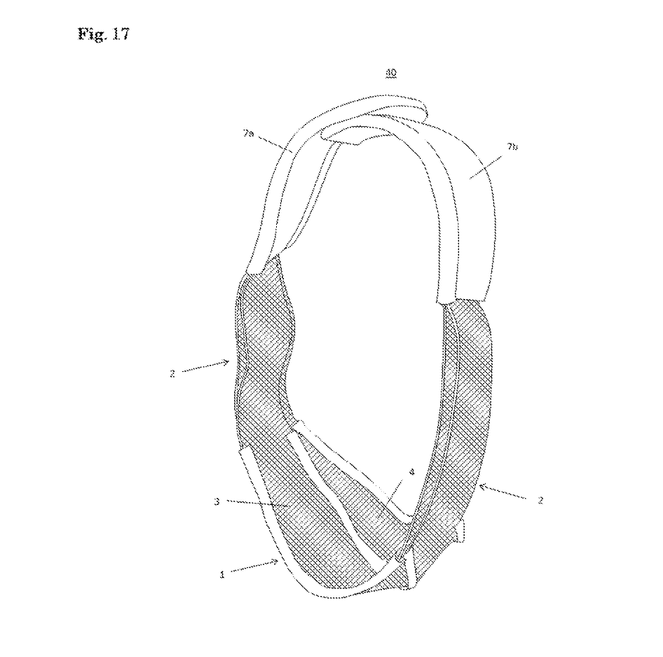

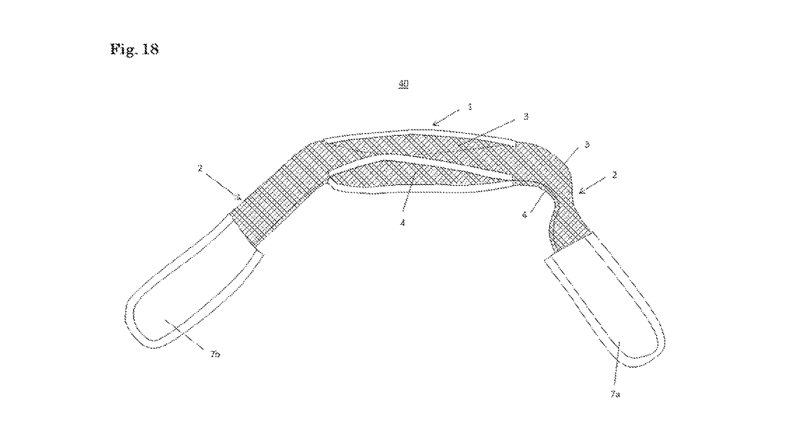

FIG. 17 is a perspective view illustrating another example 40 of the jaw fixation belt of the first embodiment to which the invention is applied, and FIG. 18 is a development schematic view of the other example 40 of the jaw fixation belt of the above embodiment.

Although, the jaw fixation belt 10 of the first embodiment includes the pair of displacement prevention belts 5, 5, the displacement prevention belt detachable members 8a, 8b provided at respective the ends of the displacement prevention belts 5, 5, and the pair of coupling belts 6, 6, as the jaw fixation belt 40 of the other example, the pair of displacement prevention belts 5, 5, the displacement prevention belt detachable members 8a, 8b provided at respective the ends of the displacement prevention belts 5, 5, and the pair of coupling belts 6, 6 may be eliminated. Eliminating the pair of displacement prevention belts 5, 5, the displacement prevention belt detachable members 8a, 8b provided at respective the ends of the displacement prevention belts 5, 5, and the pair of coupling belts 6, 6 simplifies the jaw fixation belt 40 and makes its manufacture easier.

### Second Embodiment of the Invention

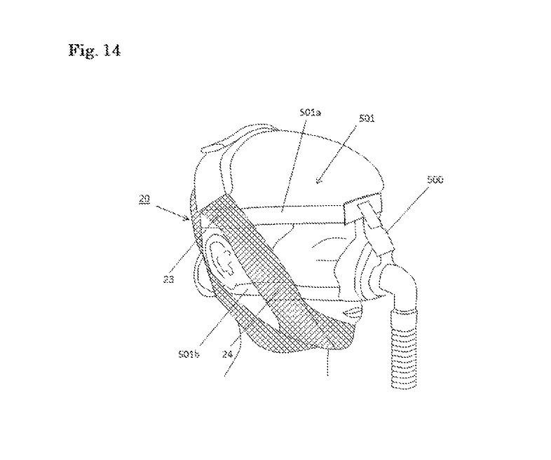

FIG. 13 is a schematic view of a state where a jaw fixation belt 20 of a second embodiment to which the invention is applied, and FIG. 14 is a perspective view of a state where the jaw fixation belt 20 of the above embodiment is fitted.

The jaw fixation belt 20 of the second embodiment of the invention is provided with a pair of holes 23, 24 that enables combination use of the jaw fixation belt 20 and a mask 500 used for treatment of sleep apnea syndrome. Since other configurations are similar to those of the jaw fixation belt 10 of the first embodiment, the same reference numerals are given to the overlapping members and structures and the overlapping description will be omitted.

The jaw fixation belt 20 includes a pair of holes 23, 24 for respectively inserting therein belts 501a, 501b of a fixture 501 for fitting a respirator in the pair of face-end mounting units 22, 22 provided in the jaw fixation belt 20.

The pair of face-end mounting unit 22, 22 is a site to be in contact with both ends of the face, and is doubly formed by overlapping the first jaw tightening belt 3 and the second jaw tightening belt 4 with each other. The holes 23, 24 are respectively provided on the side of the chin fixation portion 1 and the side of the detachable members 7a, 7b in the respective face-end mounting units 22.

The pair of the holes 23, 24 is for inserting therein the belts 501a, 501b of the fixture 501 for wearing a respirator, is sandwiched by the first jaw tightening belt 3 and the second jaw tightening belt 4, and has a width that allows the belts 501a, 501b to be respectively adequately inserted therein to be substantially perpendicular to the longitudinal direction of the face-end mounting unit 22.

The positions of the pair of the holes 23, 24 are appropriately changeable depending on the positions of the belts 501a, 502b.

As to a forming method of the pair of holes 23, 24, when both edges D1, D2 of the first jaw tightening belt 3 and the second jaw tightening belt 4 are not joined, the first jaw tightening belt 3 and the second jaw tightening belt 4 are sewed at four places of sewing places 23a, 23b, 24a, 24b, the hole 23 to form between the sewing places 23a, 23b and the hole 24 is formed between the sewing places 24a, 24b. When both edges D1, D2 of the first jaw tightening belt 3 and the second jaw tightening belt 4 are joined, a portion of not being joined is formed at each of two places of both edges D1, D2, and the places of not being joined shall be the holes 23, 24.

As to a wearing method, the belts 501a, 501b of the fixture 501 for wearing a respirator are respectively inserted into the pair of holes 23, 24 of the jaw fixation belt 20 before the mask 500 is worn on the wearer 100 to attach the fixture 501 for wearing the respirator to the jaw fixation belt 20. Then, the mask 500 is worn on the wearer 100 with the fixture 501 for wearing the respirator, and the jaw fixation belt 20 is worn on the wearer 100.

In this manner, concurrently using the jaw fixation belt 20 and the mask 500 used for treatment of sleep apnea syndrome makes it possible to firmly fix the chin to the head by the jaw fixation belt 20 to keep a mouth closed state. Therefore, the problems that occur when the mask 500 is used (dryness in the mouth, leakage of oxygen from the mouth, etc.) is overcame, it becomes possible to more comfortably perform treatment of sleep apnea syndrome.

### Third Embodiment of the Invention

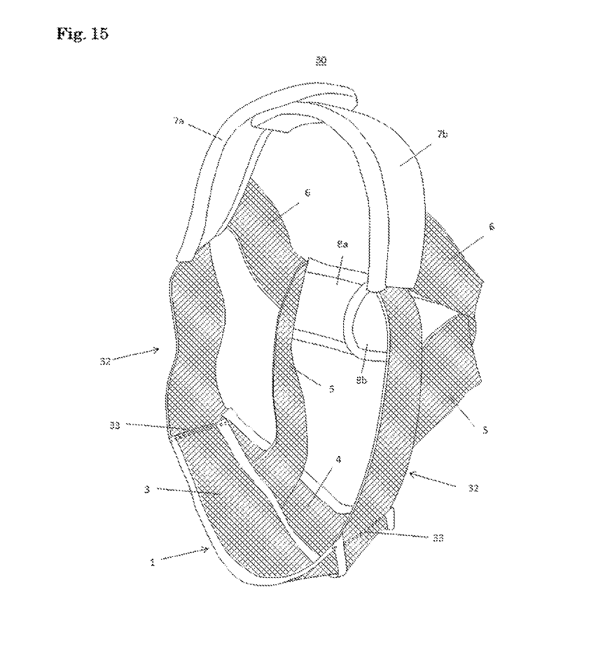

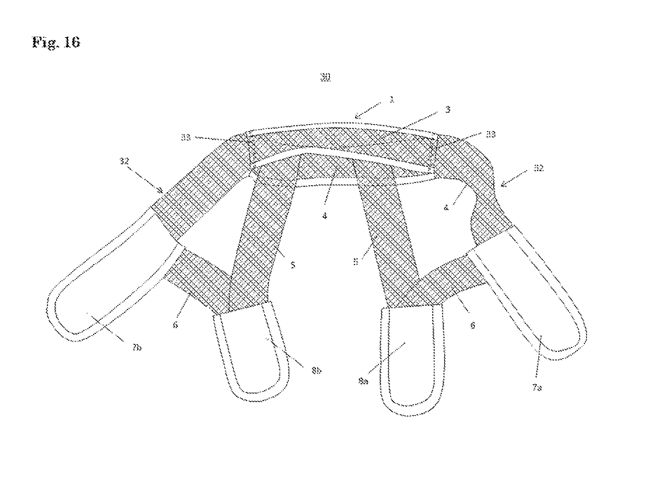

FIG. 15 is a perspective view illustrating a jaw fixation belt 30 of a third embodiment to which the invention is applied, and FIG. 16 is a schematic view of a state where the jaw fixation belt 30 of the above embodiment is spread.

In the jaw fixation belt 10 of the first embodiment, although the pair of face-end mounting units 2, 2 that is a site to be in contact with both ends of the face is doubly formed by joining the first jaw tightening belt 3 and the second jaw tightening belt 4 in such a manner that the both ends of the first jaw tightening belt 3 and the second jaw tightening belt 4 are overlapped with each other, the jaw fixation belt 30 of the third embodiment uses either the first jaw tightening belt 3 or the second jaw tightening belt 4 as face-end mounting units 32, 32. The other structures are similar to those of the jaw fixation belts 10, 20 of the first and second embodiments, so that overlapping description will be omitted.

The pair of face-end mounting units 32, 32 of the jaw fixation belt 30 of the invention is formed by utilizing the ends of the first jaw tightening belt 3 or the second jaw tightening belt 4, and the chin fixation portion 1 thereof is formed by utilizing the central parts of the first jaw tightening belt 3 and the second jaw tightening belt 4.

When the second jaw fastening belt 4 is used for the face-end mounting units 32, the chin fixation portion 1 is formed by utilizing the central parts of the first jaw tightening belt 3 and the second jaw tightening belt 4, and both ends 33, 33 of the first jaw fastening belt 3 are joined to the second jaw fastening belt 4 by thermo-compression, bonding sewing, bonding, etc.

When the first jaw fastening belt 3 is used for the face-end mounting units 32, the chin fixation portion 1 is formed by utilizing the central parts of the first jaw tightening belt 3 and the second jaw tightening belt 4, and both ends of the second jaw fastening belt 4 are joined to the first jaw fastening belt 3 by thermo-compression bonding, sewing, bonding, etc.

In this manner, a stretch force in the face-end mounting units to be worn at both ends of the face is changed/adjusted with respect to the places other than that in manufacturing stage by reduplicating the first jaw tightening belt and the second jaw tightening belt, or by using one of the first jaw tightening belt and the second jaw tightening belt as the face-end mounting units, in order to provide a jaw fixation belt that can be comfortably worn without causing the wearer to feel pain at both sides of the face.

### Experimental Example 1

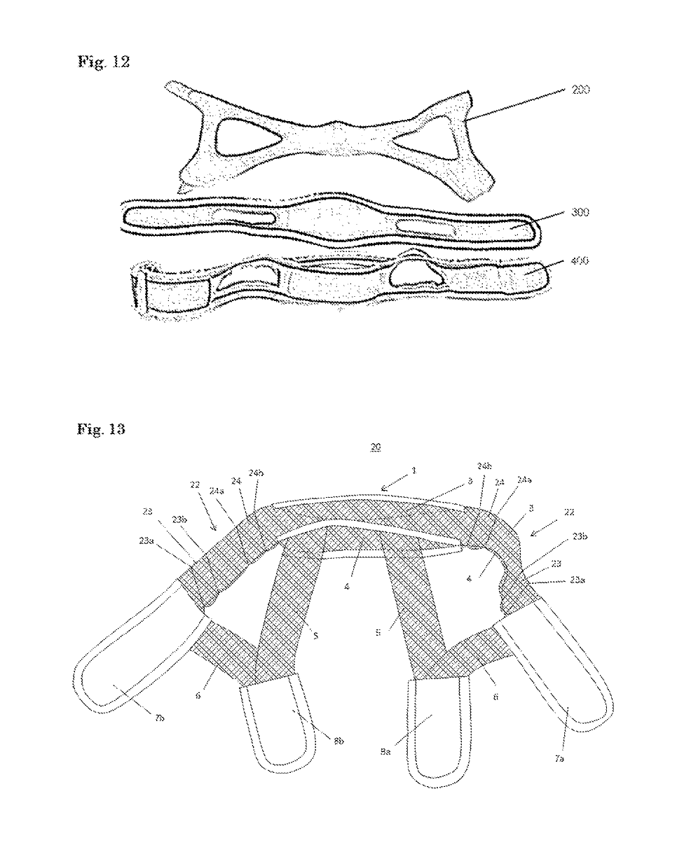

FIG. 12 is a photograph showing conventional jaw fixation belts.

Comparative experiments of stretch rate, durability, etc. were performed using the jaw fixation belts of the embodiments and the jaw fixation belts 200, 300, 400 (FIG. 12) of commercial products as comparative examples (conventional examples). Three sizes of the jaw fixation belts were prepared: S (head circumference: not more than 55 cm); M (head circumference: 56 cm to 59 cm); and L (head circumference: not less than 60 cm).

The jaw fixation belt of the embodiment was formed by a three-dimensional double sewing, in which sewing was performed such that the stacking area of the first jaw tightening belt and the second jaw tightening belt is gradually increased from the center toward the endmost portions of the chin fixation portion (FIG. 4, FIG. 6), and the endmost portions of the chin fixation portion were sewn with both edges D1, D2 being overlapped with each other such that the first jaw fixation portion and the second jaw fixation belt are not substantially displaced.

In the measuring method of the stretch rate, the constant load stretch rate at 0.5 kg load described in JIS L1096 was measured.

Is the measuring method of durability, 15 cm of cloth parts of the conventional jaw fixation belts and the jaw fixation belts of the embodiment were sampled, the width of the amplitude of each of the cloth parts was adjusted such that the surface side was bent toward the outer direction with a grip distance of 10 cm to 5 cm using a De Mattia type tension and bending fatigue tester, bending operation was repeated by 7500 times at a speed of 60 times per minute, and when an abnormality such as breakage that hinders the use was generated, it was regarded as failure, and when no abnormality was identified, it was regarded as success.

As to measuring methods of air permeability and bad smell, wearers were interviewed about how they felt in wearing the belts.

The measurement results are illustrated below (Table 1).

TABLE 1

| | Comparative Example | Jaw Fixation Belt of |
|---|---|---|
| | (Commercial Product) | The Embodiment |
| Material | Urethane Foam Bonding | Mesh Fabric Using |
| | | Covering Yarn |
| Stretch Rate | 130% | 180% |
| Air Permeability | Low | High |
| Durability | Failure in 7500 Times De | Success in 7500 Times De |
| | Mattia Durability Test | Mattia Durability Test |
| Odor | Urethane Foam Smell | Odorless |
| Sewing | One-Piece Formation | Three-Dimensional Double |
| Specification | | Sewing |
| Size Selection | Only One Size | Three Sizes of S, M, L |

Above Table 1 shows that the jaw fixation belt of the embodiment has a high stretch rate of 180% as well as high air permeability and durability as compared with the jaw fixation belt of a commercial product. Less bad smell enables comfortable use of the jaw fixation belt.

### Experimental Example 2

By using the jaw fixation belt of the embodiment and the jaw fixation belts 200, 300, 400 (FIG. 12) of commercial products as comparative examples (conventional examples), an interview about wearing feeling and the like was performed on subjects who had actually worn the belts for a predetermined period.

The results of the interview will be described below.

### Subject 1

### Sex: Male

### Age: 77 Years Old

### Period of Use: Jan. 10 to Jan. 30, 2016

### Number of Days to Wear: 21 Days

Impression: He uses the belt in order to prevent his mouth from opening, because when falling asleep, his mouth opens and his breathing comes to mouth breathing so that his mouth gets dry. The jaw fixation belts of the conventional examples have problems in that he gets sweaty because the material is made of a sponge rubber product, that the belts are readily detached due to his rolling over while sleeping, and so on. However, the jaw fixation belt of the embodiment is very easy to use. He wants to buy the jaw fixation belt of the embodiment.

### Subject 2

### Sex: Male

### Age: 26 Years Old

### Period of Use: Jan. 21 to Feb. 21, 2016

Number of Days to Wear: about 27 days
Impression: As compared with the conventional fixation belts, the jaw fixation belt of the embodiment is comfortable as he thought because the mesh fabric causes no stuffy conditions. The products of other companies (jaw fixation belts of the conventional examples) inevitably cause stuffy conditions when wearing them for a long time even in this season, so that it is uncomfortable. Effect on mouth breathing is also more than expected. He wears the jaw fixation belt of the embodiment during studying, and he can concentrate on and effectively progress his study. Facial contour has become sharp. His family also says that his facial contour has become sharp.

As described above, the jaw fixation belts of the embodiments can be used in various fields such as: for preventing snoring, sleep apnea syndrome, and the like; as a medical belt for fixing the jaw used for a gastroscopy examination by an endoscope or an endoscopic surgery of stomach or esophagus over a long time; as a supporter used during sports or martial arts; and a belt for cosmetic purposes to yield a small face effect, and can be used comfortably.

### REFERENCE NUMERALS

10, 20, 30, 40 jaw fixation belt
1 chin fixation portion
2 face-end mounting unit
3 first jaw tightening belt
4 second jaw tightening belt
5 displacement prevention belt
6 coupling belt
7a, 7b detachable member
8a, 8b displacement prevention belt detachable member
23, 24 hole
31 first cloth piece
32 third cloth piece
41 fourth cloth piece
42 second cloth piece
100 wearer

1. A jaw fixation belt worn by hanging the jaw fixation belt between the top of the head and the jaw, comprising:
a first jaw tightening belt for tightening a jaw from a front side of a chin; and
a second jaw tightening belt for tightening the jaw from a back side of the chin, wherein
the jaw fixation belt has a chin fixation portion in which one edge of a central part of the first jaw tightening belt and another edge of a central part of the second jaw tightening belt are joined.

2. The jaw fixation belt according to claim 1, wherein
the first jaw tightening belt and the second jaw tightening belt are formed of mesh fabric that is an elastic narrow-width knit created by a covering yarn whose core is a rubber-based or polyurethane elastic yarn.

3. The jaw fixation belt according to claim 1, further comprising face-end mounting units to be fitted to both ends of a face formed of the first jaw tightening belt and/or the second jaw tightening belt.

4. The jaw fixation belt according to claim 1, wherein
a first cloth piece is provided at the one edge of the central part of the first jaw tightening belt,
a second cloth piece is provided at the other edge of the central part of the second jaw tightening belt, and
the chin fixation portion is formed by joining the first cloth piece and the second cloth piece.

5. The jaw fixation belt according to claim 1, wherein
the front side of the chin contacts with the central part of the first jaw tightening belt, and the back side of the chin contacts with the central part of the second jaw tightening belt so that the chin is fixed in such a manner that the chin is sandwiched by the first jaw tightening belt and the second jaw tightening belt.

6. The jaw fixation belt according to claim 1, wherein
a third cloth piece is provided at another edge of the center of the first jaw tightening belt, and a fourth cloth piece is provided at one edge of the central part of the second jaw tightening belt.

7. The jaw fixation belt according to claim 4, wherein
the first cloth piece and the second cloth piece are formed of a material having a stretch rate smaller than that of the first jaw tightening belt and the second jaw tightening belt.

8. The jaw fixation belt according to claim 6, wherein
the third cloth piece and the fourth cloth piece are formed of a material having a stretch rate smaller than that of the first jaw tightening belt and the second jaw tightening belt.

9. The jaw fixation belt according to claim 1 wherein
the first jaw tightening belt and the second jaw tightening belt consist of a band-like member comprised of mesh fabric in which a large number of holes are formed, and the mesh fabric has a stretch rate of 120 to 300%.

10. The jaw fixation belt according to claim 1, wherein
the jaw fixation belt is further provided with a pair of displacement prevention belts attached at a place where the central part of the first jaw tightening belt and the central part of the jaw fixation belt are joined.

11. The jaw fixation belt according to claim 1, wherein the jaw fixation belt is further provided with a pair of coupling belts provided between the face-end mounting units and the displacement prevention belt.

12. The jaw fixation belt according to claim 1, wherein, the jaw fixation belt is provided with a hole for attaching a mask used for treatment of sleep apnea syndrome.
